(19) United States
(12) Patent Application Publication
(10) Pub. No.: US 2022/0409027 A1
(43) Pub. Date: Dec. 29, 2022

(54) INSERTION DEVICE

(71) Applicant: OLYMPUS CORPORATION, Tokyo (JP)

(72) Inventor: Motoko KAWAMURA, Tokyo (JP)

(73) Assignee: OLYMPUS CORPORATION, Tokyo (JP)

(21) Appl. No.: 17/899,691

(22) Filed: Aug. 31, 2022

Related U.S. Application Data
(63) Continuation of application No. PCT/JP2020/009827, filed on Mar. 6, 2020.

(51) Int. Cl. A61B 1/01; A61B 1/00; A61B 1/005

(52) U.S. Cl. CPC A61B 1/01; A61B 1/00165; A61B 1/00112; A61B 1/00071; A61B 1/0016; A61B 1/0057

(57) ABSTRACT

An endoscope as an insertion device includes an insertion portion main body extended in a direction of a longitudinal axis and having flexibility, a motor provided in an operation portion disposed on a proximal end side of the insertion portion main body, a transmission member inserted into the insertion portion main body, extended outward the insertion portion main body along the longitudinal axis, rotated around an axis by driving force of the motor, and transmitting the rotation to a distal end side of the insertion portion main body, and a detection device detecting a position along the longitudinal axis of the insertion portion main body at a predetermined site of the transmission member.

### CROSS REFERENCE TO RELATED APPLICATION

This application is a continuation application of PCT/JP2020/009827 filed on Mar. 6, 2020, the entire contents of which are incorporated herein by this reference.

### BACKGROUND OF THE INVENTION

### 1. Field of the Invention

The present invention relates to an insertion device in which a transmission member is rotated with driving force of a driving source to rotate a driven member.

### 2. Description of the Related Art

A medical endoscope generally includes an insertion portion and an operation portion positioned on a proximal end side of the insertion portion. The insertion portion is elongated along a long axis direction and is inserted into a body cavity. An image pickup optical system and an illumination optical system configuring an observation optical system are provided at a distal end portion of the insertion portion. During observation with the endoscope, the distal end portion of the insertion portion is inserted toward a site to be examined.

In addition, an endoscope has been known as well, that is provided with an insertion support function of supporting movement of the insertion portion to be inserted into a tube cavity, by disposing a structure disposed rotatably around an axis along a longitudinal direction of the insertion portion on an outer periphery of the insertion portion, and disposing a spiral protrusion on an outer peripheral surface of the structure.

In the endoscope provided with the insertion support function, for example, drive force of an electric motor disposed inside the operation portion is transmitted to a drive shaft which is a flexible driving force transmission member inserted into the insertion portion. When the driving force is transmitted to the drive shaft, the drive shaft rotates around the axis, and the rotation is transmitted to the above-described structure. Upon receiving the rotation of the drive shaft, the structure is rotated forward and backward around the axis a1ong the longitudinal direction of the insertion portion. When the spiral protrusion is in contact with a wall of the tube cavity in the rotating state of the structure, movement for operating the spiral projection backward and forward along the wall of the tube cavity, or movement for pulling the wall of the tube cavity in a longitudinal axis direction of the insertion portion by the spiral protrusion, is performed.

For example, Japanese Patent No. 6165353 discloses an endoscope apparatus provided with an insertion support function having a torque limit function of stopping rotation of a motor, when a drive current of the motor for rotating a structure reaches or exceeds a threshold value. Further, in the endoscope apparatus disclosed in Japanese Patent No. 6165353, a detection probe for detecting a bent shape of the insertion portion is disposed in the insertion portion, and a shape of the detection probe is detected by an observation device which is an external device, so that operation of the above-described torque limit function is changed according to the bent shape of the insertion portion.

### SUMMARY OF THE INVENTION

An insertion device in an aspect of the present invention includes a flexible tube extended in a direction of a longitudinal axis and having flexibility, a driving source disposed on a proximal end side of the flexible tube, a transmission member inserted into the flexible tube and extended outward from the proximal end side of the flexible tube along the longitudinal axis of the flexible tube, and configured to be rotated about an axis by driving force of the driving source and transmit the rotation to a distal end portion side of the flexible tube, and a detection device configured to detect a position, along the longitudinal axis of the flexible tube, at a predetermined site of the transmission member.

An insertion device in another aspect of the present invention includes a flexible tube extended in a direction of a longitudinal axis and having flexibility, a driving source disposed on a proximal end side of the flexible tube, a transmission member inserted into the flexible tube and extended outward from the proximal end side of the flexible tube along the longitudinal axis of the flexible tube, and configured to be rotated about an axis by driving force of the driving source and transmit the rotation to a distal end portion side of the flexible tube, a sheath disposed so as to cover an outer periphery of the transmission member, and a detection device configured to detect a relative position, along the direction of the longitudinal axis of the flexible tube, of each of a predetermined site of the sheath and a predetermined site of the transmission member.

### BRIEF DESCRIPTION OF THE DRAWINGS

FIG. 1 is a schematic diagram illustrating an endoscope system in an aspect of the present invention.

FIG. 2 is a diagram for explaining an operation portion of an endoscope included in the endoscope system.

FIG. 3 is a diagram for explaining a driving unit inside a driving source housing portion provided in the operation portion.

FIG. 4A is a diagram for explaining a positional relationship between a magnet and a proximal end surface of a driving force receiving portion in a straight state of a flexible tube.

FIG. 4B is a diagram for explaining a positional relationship between the magnet and the proximal end surface of the driving force receiving portion in a state where the flexible tube is bent.

FIG. 4C is a diagram for explaining a positional relationship between the magnet and the proximal end surface of the driving force receiving portion in a state where the flexible tube is bent at an angle larger than a prescribed angle.

FIG. 4D is a diagram for explaining a positional relationship between the magnet disposed at each of multiple detection ranges included in a detection device and the proximal end surface of the driving force receiving portion.

FIG. 5 is a diagram for explaining another configuration example of an insertion device.

### DETAILED DESCRIPTION OF THE PREFERRED EMBODIMENT(S)

Hereinafter, an embodiment of the present invention will be described with reference to the figures.

Note that, in each of the figures used in the following description, in order to set a size of each constituent element to be recognizable in the figure, a scale is varied for each constituent element in some cases. That is, the present invention is not limited only to the number of constituent elements, shapes of the constituent elements, ratios of sizes of the constituent elements, and a relative positional relationship among the constituent elements described in the figures.

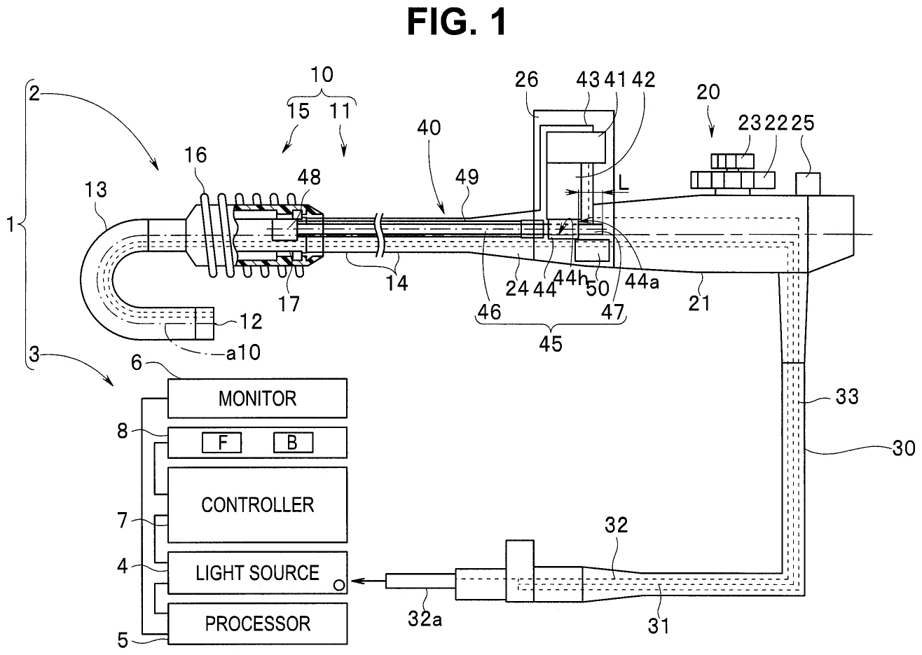

In the present embodiment, an insertion device is an endoscope system 1 illustrated in FIG. 1. The endoscope system 1 includes an endoscope 2 and a control system 3. The control system 3 includes multiple units 4, 5, 7, and 8 connected to the endoscope 2.

Although the endoscope 2 is used to explain an insertion instrument in the present embodiment, the present invention is a technique also applicable to, instead of the endoscope 2, a catheter, other insertion instruments to be inserted into a living body, or the like.

The control system 3 includes a light source unit 4, a processor 5, a monitor 6, a controller 7, an input unit 8, and the like. The light source unit 4 includes a light source that emits illumination light. The processor 5 processes an image. The monitor 6 displays an image. The controller 7 has a function as an operating portion, a function as a determination portion, and the like. The controller 7 having such functions controls the endoscope system 1 as a whole.

In the present embodiment, the input unit 8 is a foot switch. For example, a forward switch F and a backward switch B are disposed on the foot switch 8. The forward switch F and the backward switch B constitute an instruction portion. A signal outputted from the instruction portion of the foot switch 8 is inputted to the controller 7. The controller 7 is configured to control a driving unit 40 to be described later provided in the endoscope 2, based on the signal outputted from the instruction portion.

Note that the input unit 8 is not limited to a foot switch and may be a keyboard, a switch at hand, or the like.

The controller 7 is not limited to a dedicated device, and for example, a general-purpose processing device such as a personal computer in which an arbitrary program is installed may be used.

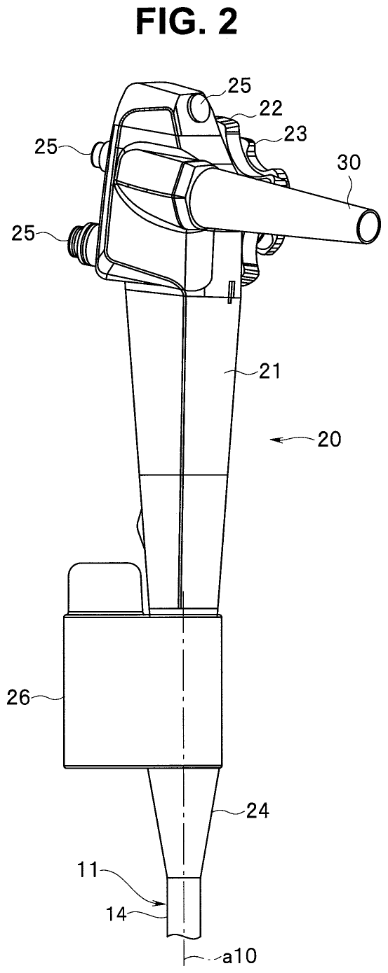

The endoscope 2 illustrated in FIG. 1 and FIG. 2 includes an insertion portion 10, an operation portion 20, and a universal cable 30. The insertion portion 10 is elongated and inserted into a tube cavity which is an object. The operation portion 20 is disposed on a proximal end side of the insertion portion 10. The universal cable 30 is extended from the operation portion 20.

The endoscope 2 includes the driving unit 40 from the operation portion 20 to the insertion portion 10. Further, the endoscope 2 is connected to the control system 3 with the universal cable 30.

An image signal cable 31 and an illumination optical system 32 such as a fiber bundle are inserted through the insertion portion 10. the operation portion 20, and the universal cable 30 of the endoscope 2. A reference numeral 32a denotes a light guide connector of the illumination optical system 32, and a reference numeral 33 denotes an electric cable 33 extended from the driving unit 40. The image signal cable 31 and the electric cable 33 are each connected to the processor 5 and the controller 7, via a light source 4. Note that the electric cable 33 may be arranged outside the universal cable 30.

The insertion portion 10 includes an insertion portion main body 11 that is elongated with respect to a longitudinal axis a10, which is an axis in a longitudinal direction, and a spiral tube 15. The spiral tube 15 has a spiral fin 16 formed on an outer periphery of a cylindrical member and is disposed on an outer peripheral surface on a distal end portion side of the flexible tube 14. Note that the spiral tube 15 may be configured to be attachable to and detachable from the insertion portion main body 11 so as to be an independent configuration separate from the insertion portion 10.

The insertion portion main body 11 includes a rigid distal end portion 12, a bending portion 13, and the flexible tube 14 in order from a distal end side. The flexible tube 14 has flexibility, thus can follow a curve of the tube cavity.

The operation portion 20 includes a grasping portion 21 to be grasped by a user. The bending portion 13 can be bent in directions corresponding to four directions of up, down, left, and right in an observation image displayed on the monitor 6 in accordance with operations of knobs 22 and 23 provided on the grasping portion 21. Structure of the bending portion 13 is well known, and a detailed description thereof will be omitted.

The bending portion 13 is bent downward or upward by a clockwise or counterclockwise operation of the first knob 22. Further, the bending portion 13 is bent rightward or leftward by a clockwise or counterclockwise operation of the second knob 23.

The distal end portion 12 is provided with an observation optical portion (not illustrated), a cleaning nozzle (not illustrated), a channel distal end opening (not illustrated), and the like. The observation optical portion is connected to the image signal cable 31. Liquid or gas is ejected from the cleaning nozzle. The channel distal end opening is an opening on a distal end side of a treatment instrument insertion channel (not illustrated) through which a forceps or the like is inserted.

A reference numeral 24 denotes a bend preventing member. The bend preventing member 24 supports a proximal end of the flexible tube 14 and prevents bending at a boundary portion between the operation portion 20 and the insertion portion 10.

Note that, in addition to the knobs 22 and 23, a switch 25 to which various instructions are assigned is disposed on the grasping portion 21. One or more switches 25 are provided. The switch 25 may include not only an electrical switch but also a mechanical switch such as a suction button or a gas feeding/liquid feeding button. A reference numeral 26 denotes a driving source housing portion. The driving source housing portion 26 is provided at a predetermined position of the grasping portion 21. Although not illustrated, a channel proximal end opening is provided on a proximal end side of the driving source housing portion 26.

The spiral tube 15 is positioned closer to a proximal end side than the bending portion 13, and on an outer peripheral surface in a vicinity of a distal end of the flexible tube 14. The spiral tube 15 rotates clockwise or counterclockwise about the longitudinal axis a10 of the insertion portion 10.

When configured to be attachable to and detachable from the insertion portion main body 11, the spiral tube 15 causes the distal end portion 12 and the bending portion 13 to pass therethrough from a distal end side of the insertion portion main body 11 and is detachably attached to the above-described position of the flexible tube 14.

When driving force of the driving unit 40 is transmitted, the spiral tube 15 rotates with respect to the insertion portion main body 11, thereby assisting insertion or removal of the insertion portion 10 into or from the tube cavity.

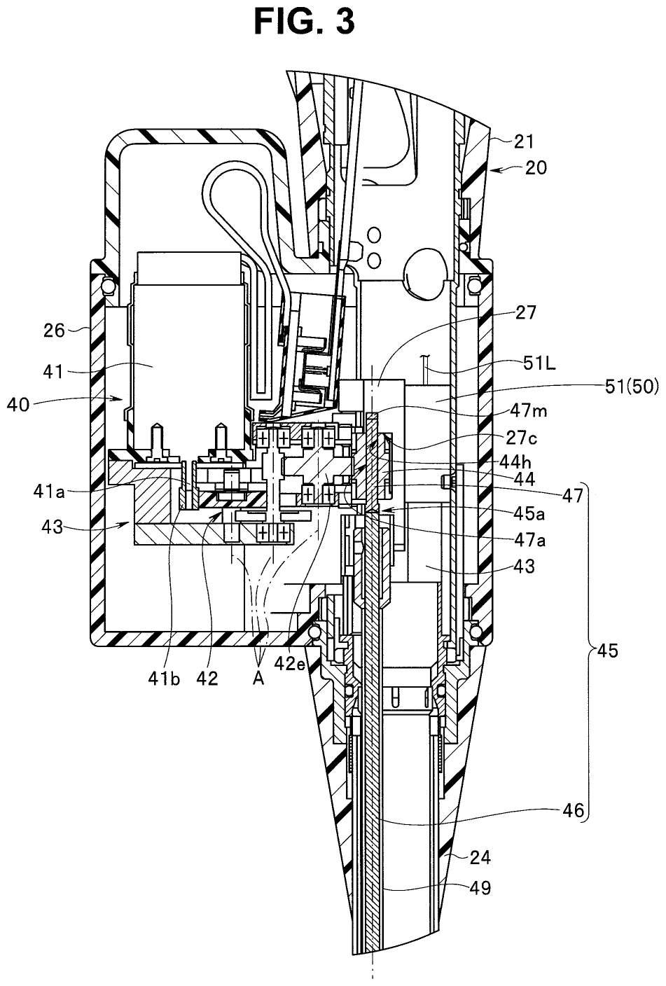

The driving unit 40 will be described with reference to FIG. 1 and FIG. 3.

The driving unit 40 mainly includes an electric motor (hereinafter, abbreviated as a motor) 41 and a transmission member 45. The motor 41 is a driving source. An output shaft 41a of the motor 41 rotates clockwise or counterclockwise. Driving force of the motor 41 is transmitted to a gear portion 42 including at least one gear meshing with a motor gear 41b fixed to the output shaft 41a, and a driving force receiving portion 44, and then transmitted to the transmission member 45.

The gear portion 42 and the motor 41 are held by a casing 43. The casing 43 is fixed to a frame (not illustrated) provided inside the driving source housing portion 26.

The driving force receiving portion 44 is a cylindrical member having an axial through-hole 44h. A tooth portion that meshes with the gear of the gear portion 42 is provided on an outer peripheral surface of the driving force receiving portion 44. The driving force receiving portion 44 is an immobile member. To be specific, the driving force receiving portion 44 is disposed inside a recessed portion 27c of a partition member 27 fixed inside the grasping portion 21. The driving force receiving portion 44 disposed inside the recessed portion 27c does not slide in a direction of the longitudinal axis a10 and is held so as to rotate clockwise or counterclockwise about the longitudinal axis a10. That is, the driving force receiving portion 44 is rotated such that a disposition position thereof in the direction of the longitudinal axis a10 is not changed in the grasping portion 21.

The transmission member 45 includes a drive shaft 46 and a rotating member 47. The drive shaft 46 is a stranded wire formed by stranding multiple wires. The drive shaft 46 has predetermined elasticity, flexibility, and torque transmission properties. The rotating member 47 is a rigid rod-like member.

In the transmission member 45, an end portion on a proximal end side of the drive shaft 46 and an end portion on a distal end side of the rotating member 47 are integrally configured. In the transmission member 45, an axis of the drive shaft 46 and an axis of the rotating member 47 are configured to be coaxial.

The drive shall 46 is inserted mainly into the flexible tube 14 along the longitudinal axis a10 of the insertion portion 10. A driving force output portion 48 is fixed to an end portion on a distal end side of the drive shaft 46. A transmission target portion 17 provided on the spiral tube 15 is joined to the driving force output portion 48.

The proximal end side of the drive shaft 46 is extended along the longitudinal axis a10 of the insertion portion 10 from a proximal end side of the flexible tube 14, passes through an inside of the bend preventing member 24 of the operation portion 20, and is guided into the grasping portion 21. The rotating member 47 and the drive shaft 46 are connected to each other in a vicinity of the bend preventing member 24 inside the grasping portion 21.

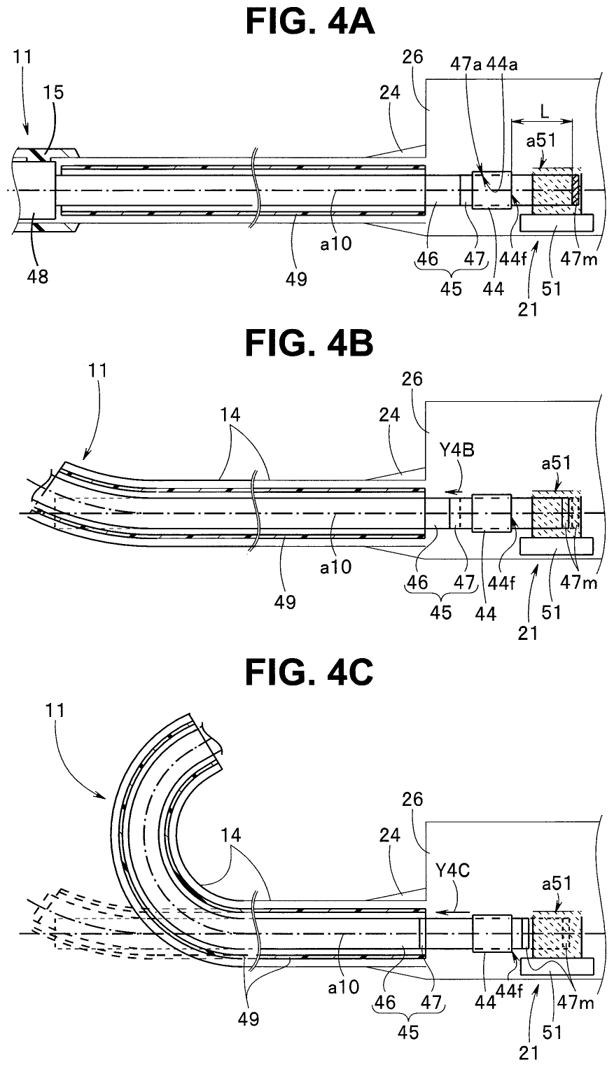

As illustrated in FIG. 1 and FIG. 4A, the rotating member 47 is extended along the longitudinal axis a10 into the grasping portion 21 of the operation portion 20. The rotating member 47 passes through the axial through-hole 44h of the driving force receiving portion 44, and protrudes from a proximal end surface 44f of the driving force receiving portion 44 along the direction of the longitudinal axis a10 by a predetermined length L.

In FIG. 4A, a reference numeral 47m denotes a magnet. The magnet 47m is fixed to a proximal end surface of the rotating member 47.

An intermediate portion of the rotating member 47 is a rotation transmitting portion 47a. Rotation of the driving force receiving portion 44 is transmitted from a through-hole transmission portion 44a provided in the axial through-hole 44h to the rotation transmitting portion 47a to rotate the rotating member 47. Then, the rotating member 47 is disposed slidably in an axial direction in the axial through-hole 44h of the rotation transmitting portion 47a.

On an outer peripheral surface side of the drive shaft 46, a sheath 49 for protecting the drive shaft 46 is provided. The sheath 49 is formed of a resin material having electrical insulation, and wear resistance and flexibility. An end portion on a proximal end side of the sheath 49 is fixed to a distal end side of the bend preventing member 24. An end portion on a distal end side of the sheath 49 is fixed at a predetermined position on a distal end side of the flexible tube 14.

A reference numeral 50 denotes a detection device. In the present embodiment, the detection device 50 is a magnetic sensor 51. The magnetic sensor 51 is fixed to a partition plate 27. The magnetic sensor 51 detects whether or not the magnet 47m moving along the direction of the longitudinal axis a10 is positioned within a detection range a51 of the magnetic sensor 51 indicated by broken lines. When detecting the magnet 47m within the detection range a51, the magnetic sensor 51 transmits a detection signal to the controller 7 with a signal line 51L.

It is prescribed that when the flexible tube 14 is in a straight state illustrated in FIG. 4A, the magnet 47m is disposed within the detection range a51 of the magnetic sensor 51. The magnetic sensor 51 outputs a detection signal to the controller 7 when the magnet 47m is positioned within the detection range a51. Detection sensitivity of the magnetic sensor 51 can be adjusted by changing a size (thickness or the like) of the magnet 47m.

In addition, in the present embodiment, the gear portion 42 is a gear train in which the multiple gears are arrayed. Driving force of the motor 41 is transmitted to the motor gear 41b, the gear train, the driving force receiving portion 44, and the transmission member 45 in order. Gear ratios of the multiple gears provided in the gear train are appropriately set, and the transmission member 45 is driven at predetermined torque and a predetermined speed.

Note that the gear train can be made unnecessary depending on a type of the motor 41 and a control method of the motor 41. That is, depending on a type of the motor 41 or a control method, it is also possible to transmit the driving force of the motor 41 to one gear, or directly to the driving force receiving portion 44 to drive the transmission member 45, without using a gear train in which multiple gears are arrayed.

Operation of the above-described endoscope system 1 will be described.

An operator inserts the insertion portion main body 11 of the endoscope into the tube cavity from an inlet of the tube cavity. The operator, while the insertion portion main body 11 is inserted, operates the foot switch 8, as necessary.

When the forward switch F is operated by the operator, the magnetic sensor 51 is brought into an operating state by the controller 7. While the magnetic sensor 51 detects the magnet 47m provided on the rotating member 47, a detection signal is outputted from the magnetic sensor 51 to the controller 7. Upon receiving the detection signal from the magnetic sensor 51, the controller 7 determines starting of motor driving, and control the motor 41 to drive.

Then, the output shaft 41a of the motor 41 rotates in a predetermined direction. The rotation of the output shaft 41a is transmitted from the motor gear 41b to the gear portion 42 and is transmitted from a rear-stage gear (see a reference numeral 42e in FIG. 3) of the gear train included in the gear portion 42 to the driving-force receiving portion 44, and the driving force receiving portion 44 rotates.

In association with the rotation of the driving force receiving portion 44, the rotating member 47 rotates, and the rotating member 47 and the drive shaft 46 rotate. The rotation of the drive shaft 46 is transmitted to the transmission target portion 17 joined to the driving force output portion 48 provided on the shaft 46. As a result, the spiral tube 15 rotates in a predetermined direction around the longitudinal axis a10 of the insertion portion 10.

As the spiral tube 15 rotates, the fin 16 also rotates about the longitudinal axis a10. When the rotating fin 16 is in contact with an inner wall surface of the tube cavity, the inner wall surface is pulled toward the proximal end side of the insertion portion 10 by the fin 16. In other words, the distal end portion 12 of the insertion portion 10 moves toward a deep portion of the tube cavity.

When the insertion portion 10 is inserted into the deep portion of the tube cavity in association with the rotation of the spiral tube 15, the flexible tube 14 of the insertion portion main body 11 is bent along a bent state of the tube cavity.

When the flexible tube 14 is changed from the straight state to a bent state at a gentle angle as illustrated in FIG. 4B, the transmission member 45 is slightly pulled into the flexible tube 14 as illustrated by an arrow Y4B. At the time, the magnet 47m fixed to the rotating member 47 also moves along the longitudinal axis a10 from a position indicated by broken lines to a position indicated by solid lines on a side of the proximal end surface 44f of the driving force receiving portion 44.

When a position of the moved magnet 47m is within the detection range a51 of the magnetic sensor 51, the magnetic sensor 51 continues to output a detection signal to the controller 7. While the detection signal from the magnetic sensor 51 is inputted to the controller 7, the controller 7 determines that the bent state of the flexible tube 14 is within a prescribed range and causes the rotation of the spiral tube 15 to continue.

On the other hand, when an integrated value of bending angles of a bent portion exceeds a prescribed value, and the flexible tube 14 is bent in a complicated manner as illustrated in FIG. 4C as compared with the bent state in FIG. 4B, the transmission member 45 is largely pulled into the flexible tube 14 as illustrated by an arrow Y4C. At the time, the magnet 47m is moved to the side of the proximal end surface 44f of the driving force receiving portion 44 along the longitudinal axis a10 and gets out of the detection range a51 of the magnetic sensor 51, and the output of a detection signal from the magnetic sensor 51 to the controller 7 is stopped.

When the output of a detection signal from the magnetic sensor 51 to the controller 7 is stopped, the controller 7, which has determined controlling of the motor, determines stopping of the motor driving, and switches to control for stopping the motor 41.

In the present embodiment, when the magnet 47m fixed to the rotating member 47 of the transmission member 45 is pulled toward the side of the proximal end surface 44f of the driving force receiving portion 44 along the longitudinal axis a10, and gets out of the detection range a51 of the magnetic sensor 51 as described above, the controller 7 determines that the integrated value of the bending angles exceeds the prescribed value and the flexible tube 14 is bent in a complicated manner, and causes the torque limit function to operate to stop the rotation of the spiral tube 15.

Note that when the backward switch B is operated by the operator, the controller 7 does not bring the magnetic sensor 51 into the operating state, and the output shaft 41a of the motor 41 is rotated in a direction opposite to that when the forward switch F is operated.

As described above, the rotation of the output shaft 41a is transmitted from the motor gear 41b to the gear portion 42, the rear-stage gear 42e of the gear train, the driving force receiving portion 44, the rotating member 47, and the drive shaft 46. Then, as described above, the rotation of the drive shaft 46 is transmitted from the driving force output portion 48 to the transmission target portion 17.

As a result, the spiral tube 15 is rotated about the longitudinal axis a10 of the insertion portion 10 in a direction opposite to that when the forward switch F is operated. At the time, the fin 16 also rotates together with the spiral tube 15. Then, in a state where the spiral tube 15 is rotating, and in a state where the fin 16 is in contact with the inner wall surface of the tube cavity, the inner wall surface is entangled with the fin 16 and pulled toward a distal end side of the insertion portion 10. In other words, the distal end portion 12 of the insertion portion 10 is moved in an opposite direction in the tube cavity, that is, from deep inside toward a direction of the inlet of the tube cavity.

The endoscope 1 in which the spiral tube 15 is disposed on a side of the bending portion 13 of the flexible tube 14 of the insertion portion main body 11 described above, has the torque limit function in which the magnetic sensor 51 fixed in the grasping portion 21 of the operation portion 20, and the magnet 47m fixed to the transmission member 45 slidable in the direction of the longitudinal axis a10 in the grasping portion 21 are disposed.

In a state where the forward switch F is operated, and when the magnet 47m is positioned within the detection range a51 of the magnetic sensor 51, the magnetic sensor 51 outputs a detection signal to the controller 7. The controller 7 that receives the detection signal controls driving of the motor 41. In a state where the motor 41 is caused to drive, and when the magnet 47m gets out of the detection range a51 of the magnetic sensor 51, the output of a detection signal to the controller 7 is stopped, the driving of the motor 41 by the controller 7 is stopped, and the rotation of the spiral tube 15 is stopped.

In the torque limit function of the endoscope 1 of the present embodiment, the magnetic sensor 51 and the magnet 47m fixed to the transmission member 45 slidable in the direction of the longitudinal axis a10 in the grasping portion 21 are disposed in the grasping portion 21 of the operation portion 20. Accordingly, it is not necessary to provide a sensor and a signal line for the torque limit function in the insertion portion main body 11. Thus, a problem that an outer diameter of the insertion portion main body 11 is increased is solved. As a result, it is possible to decrease the diameter of the insertion portion main body 11 in which the spiral tube 15 is disposed.

In addition, when the bent state of the flexible tube 14 changes, an amount of the transmission member 45 that is pulled changes. When the transmission member 45 is pulled into the flexible tube 14 and the magnet 47m fixed to a proximal end gets out of the detection range a51 of the magnetic sensor 51, and the output of a detection signal is stopped, the controller 7 determines that the bent state of the flexible tube 14 exceeds the prescribed range regardless of a bent shape of the insertion portion main body 11, and causes the torque limit function to operate.

In other words, when the magnet 47m fixed to the pulled transmission member 45 is within the detection range a51 of the magnetic sensor 51, driving force of the motor 41 can be transmitted from the transmission member 45 to the spiral tube 15 to rotate the spiral tube 15 regardless of the bent shape of the insertion portion main body 11, thereby maintaining good insertion performance.

Note that, in the above-described embodiment, when the magnet 47m is positioned within the detection range a51 of the magnetic sensor 51, a detection signal is outputted from the magnetic sensor 51 to the controller 7 to control the motor 41.

A detection range a51A of a magnetic sensor 51A illustrated in FIG. 4D includes multiple detection ranges a1, a2, and a3 along the longitudinal axis a10. The magnetic sensor 51A outputs a different detection signal for each of the detection ranges a1, a2 and a3 to the controller 7. The controller 7 controls a drive current of the motor 41 with a drive current of a preset current value, for each inputted detection signal.

To be specific, when the flexible tube 14 is in the straight state, the magnet 47m indicated by solid lines is positioned within the first detection range a1. As described above, when the forward switch F is operated and the magnetic sensor 51A is brought into the operating state, a first detection signal is outputted from the magnetic sensor 51A to the controller 7. Upon receiving the first detection signal, the controller 7 determines driving of the motor, and supplies a predetermined first drive current to the motor 41 to cause the motor 41 to be driven.

When the flexible tube 14 is bent, the magnet 47m moves along the longitudinal axis a10 into the second detection range a2 as indicated by broken lines. At the time, a second detection signal is outputted from the magnetic sensor 51A to the controller 7. Upon receiving the second detection signal, the controller 7 determines changing of driving force of the motor 41 and supplies a predetermined second drive current to the motor 41 to control the motor 41. A current value of the second drive current is preset to be higher than a current value of the first drive current.

When the flexible tube 14 is further bent, the magnet 47m moves along the longitudinal axis a10 into the third detection range a3 as indicated by two-dot chain lines. At the time, a third detection signal is outputted from the magnetic sensor 51A to the controller 7. Upon receiving the third detection signal, the controller 7 determines changing of the driving force of the motor 41 and supplies a third drive current preset to be higher than the current value of the second drive current to the motor 41, to control the motor 41.

Then, when the magnet 47m gets out of the third detection range a3 of the magnetic sensors 51A to the side of the proximal end surface 44f of the driving force receiving portion 44, the output of a detection signal from the magnetic sensor 51A to the controller 7 is stopped. As a result, as described above, the controller 7 switches the control from the control for causing the motor 41 to drive to the control for causing the motor 41 to stop.

Upon receiving the various detection signals outputted from the magnetic sensor 51A, the controller 7 outputs the predetermined drive currents corresponding to the bent state of the flexible tube 14 to the motor 41 to control the motor 41. As a result, upon receiving an optimum drive current according to the bent state of the flexible tube 14, regardless of the bent shape of the flexible tube 14, the spiral tube 15 is rotated, and stops rotating when the bent state exceeds the prescribed range.

Note that the number of detection ranges of the magnetic sensor 51A is not limited to three and may be more than three or may be two. In addition, the detection device 50 is not limited to the magnetic sensors 51 and the 51A and may be a transmissive type or reflective type optical sensor. Further, the detection device 50 is not limited to a non-contact type sensor and may be a contact type switch such as a limit switch provided with a micro-switch.

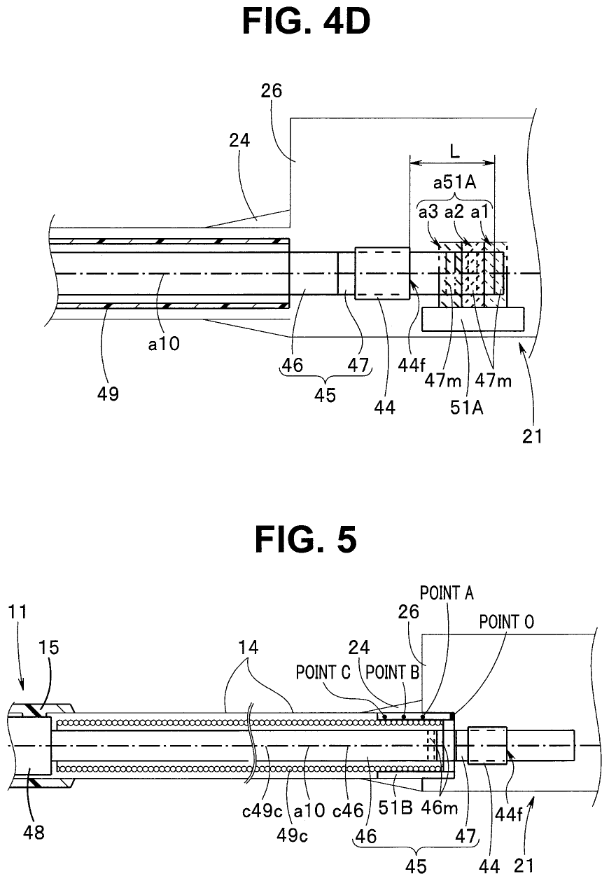

As illustrated in FIG. 5, a coil sheath 49c is provided on the outer peripheral surface side of the drive shaft 46 in place of the sheath 49. The coil sheath 49c is formed of a non-magnetic material having abrasion resistance and elasticity for protecting the drive shaft 46.

An end portion on a distal end side of the coil sheath 49c is fixed to a predetermined position on the distal end side of the flexible tube 14. An end portion on a proximal end side of the coil sheath 49c is fixed to the distal end side of the bend preventing member 24. The coil sheath 49c having elasticity is configured such that when the flexible tube 14 is bent, a length of a sheath central axis c49c increases along with an increase in an amount of bending.

As described above, the drive shaft 46 is a stranded wire formed by stranding multiple wires. In the drive shaft 46, which is a stranded wire, when the flexible tube 14 is bent, a length of a shaft central axis c46 hardly changes. Then, in the straight state of the flexible tube 14, the shaft central axis c46 and the sheath central axis c49c substantially coincide with the longitudinal axis a10.

The magnet 46m is fixed to a vicinity of a portion connected to the rotating member 47 positioned on a proximal end side of the drive shaft 45. A reference numeral 51B denotes a magnetic sensor. The magnetic sensor 51B has a function of detecting a moving distance of the magnet 46m in a non-contact manner.

The magnetic sensor 51B has three detection ranges. A first detection range is from a point O to a point A, a second detection range is from the point A to a point B, and a third detection range is from the point B to a point C. Note that the number of detection ranges is not limited to three and may be more or less than three. As described above, detection sensitivity of the magnetic sensor 51B can be adjusted by changing the size of the magnet 46m.

In the present embodiment, when the flexible tube 14 is in the straight state, the magnet 46m is positioned at a proximal end of the coil sheath 49c and is within the first detection range. When the flexible tube 14 changes from the straight state to the bent state, the length of the sheath central axis c49c increases compared to the length of the shaft central axis c46. As a result, the magnet 46m fixed to the drive shaft 46 is pulled into the coil sheath 49c along the longitudinal axis a10 as indicated by broken lines. An amount of the magnet 46m pulled, that is, a moving distance, is detected by the magnetic sensor 51B, and outputted to the controller 7.

The magnetic sensor 51B outputs the first detection signal when the magnet 46m is positioned within the first detection range along the longitudinal axis a10, outputs the second detection signal when the position is within the second detection range, and outputs the third detection signal when the position is within the third detection range. Then, when the point C is exceeded, the output of a detection signal is stopped.

In the present embodiment, when the flexible tube 14 is in the straight state, the magnet 46m is positioned at the proximal end of the coil sheath 49c between the point O and the point A.

When the forward switch F is operated, the magnetic sensor 51B is brought into the operating state by the controller 7. When the magnetic sensor 51B is operated, and when the magnet 46m is positioned within the first detection range, the first detection signal is outputted from the magnetic sensor 51B to the controller 7. On the other hand, when the magnet 46m is positioned within the second detection range, the second detection signal is outputted from the magnetic sensor 51B to the controller 7.

Upon receiving the detection signal, the controller 7 determines starting of detection of a moving distance and start motor driving. The controller 7 supplies the first drive current or the second drive current, which is a drive current corresponding to the detection signal, to the motor 41 to cause the motor 41 to drive.

When the coil sheath 49c is bent, the magnet 46m is pulled into the coil sheath 49c as indicated by an arrow Y5. A moving distance of the magnet 46m in the direction of the longitudinal axis a10 at the time is measured by the magnetic sensor 51B.

The magnetic sensor 51B outputs the first detection signal to the controller 7 until the magnet 46m exceeds the point A, outputs the second detection signal to the controller 7 until the point B is exceeded, and outputs the third detection signal to the controller 7 until the point C is exceeded.

When a different detection signal is inputted in a motor driving state, the controller 7 determines changing of driving force of the motor 41 and supplies a drive current different from the drive current supplied to the motor 41 in the driving state to control the motor 41.

Then, when the magnet 46m exceeds the point C, the magnetic sensor 51B stops the output of a detection signal from the magnetic sensor 51B to the controller 7. As a result, the controller 7 switches the control from the control for causing the motor 41 to drive to the control for causing the motor 41 to stop.

According to the configuration, the controller 7 receives a detection signal outputted from the magnetic sensor 51B, determines a relative position of each of the coil sheath 49c and the magnet 46m, and outputs an optimum drive current to the motor 41 regardless of the bent state of the flexible tube 14 to control the motor 41. As a result, the spiral tube 15 is rotated with an optimum drive current in accordance with a bent state of the coil sheath 49c regardless of the bent shape of the flexible tube 14. Then, when the magnet 46m exceeds the point C, and the bent shape of the coil sheath 49c is deformed such that a bent angle exceeds a prescribed range, or when the flexible tube 14 is bent in a complicated manner, the rotation is stopped.

Other configurations are similar to those of the above-described embodiment, and the same members are denoted by the same reference numerals and description thereof will be omitted.

The present invention is not limited to the embodiments described above, and various changes or applications can be made within the scope without departing from the spirit of the present invention.

What is claimed is:

1. An insertion device, comprising:
a flexible tube extended in a direction of a longitudinal axis and having flexibility;
a driving source disposed on a proximal end side of the flexible tube;
a transmission member inserted into the flexible tube and extended outward from the proximal end side of the flexible tube along the longitudinal axis of the flexible tube, and configured to be rotated about an axis by driving force of the driving source and transmit the rotation to a distal end portion side of the flexible tube; and
a detection device configured to detect a position, along the longitudinal axis of the flexible tube, at a predetermined site of the transmission member.

2. The insertion device according to claim 1, wherein
the transmission member transmits the rotation to a driven member disposed on the distal end portion side of the flexible tube.

3. The insertion device according to claim 1, comprising:
a determination unit configured to determine a bent state of the flexible tube based on a detection result of the detection device.

4. The insertion device according to claim 3, wherein
the detection device, when detecting that the predetermined site of the transmission member is at a predetermined position with respect to a position along the longitudinal axis of an immobile member arranged on the proximal end side of the flexible tube, outputs a detection signal to the determination unit.

5. The insertion device according to claim 4, wherein
the predetermined site of the transmission member is a proximal end portion along the longitudinal axis of the transmission member.

6. The insertion device according to claim 5, wherein
the detection device detects that the proximal end portion of the transmission member comes dose to an immobile member disposed at a proximal end portion of the flexible tube up to a predetermined distance.

7. The insertion device according to claim 1, wherein
the transmission member includes a stranded wire having elasticity flexibility, and torque transmission properties predetermined by stranding wires.

8. The insertion device according to claim 1, wherein
an endoscope is configured by disposing an operation portion at a proximal end portion of the flexible tube.

9. An insertion device, comprising:
a flexible tube extended in a direction of a longitudinal axis and having flexibility;
a driving source disposed on a proximal end side of the flexible tube;
a transmission member inserted into the flexible tube and extended outward from the proximal end side of the flexible tube along the longitudinal axis of the flexible tube, and configured to be rotated about an axis by driving force of the driving source and transmit the rotation to a distal end portion side of the flexible tube;
a sheath disposed so as to cover an outer periphery of the transmission member; and
a detection device configured to detect a relative position, along the direction of the longitudinal axis of the flexible tube, of each of a predetermined site of the sheath and a predetermined site of the transmission member.

10. The insertion device according to claim 9, wherein
the transmission member transmits the rotation to a driven member disposed on the distal end portion side of the flexible tube.

11. The insertion device according to claim 9, comprising:
a determination unit configured to determine a bent state of the flexible tube based on a detection result of the detection device.

12. The insertion device according to claim 9, wherein
the transmission member has elasticity, flexibility, and torque transmission properties predetermined by stranding wires.

13. The insertion device according to claim 12, wherein
the sheath is a coil disposed on an outer peripheral surface side of the transmission member and obtained by being wound around.

14. The insertion device according to claim 13, wherein
the detection device detects a relative position of each of the sheath and the predetermined site of the transmission member along a long axis direction.

15. The insertion device according to claim 14, wherein
the detection device detects a relative moving distance in the direction of the longitudinal axis when the predetermined site of the transmission member is pulled into the sheath.

16. The insertion device according to claim 9, wherein
a distal end portion of each of the transmission member and the sheath is fixed to the flexible tube.

17. The insertion device according to claim 9, wherein
a position in the direction of the longitudinal axis on a proximal end side of the sheath is fixed with respect to the flexible tube, and
a position in the direction of the longitudinal axis on a proximal end side of the transmission member is displaceable with respect to the flexible tube.

18. The insertion device according to claim 17, comprising:
a detection device configured to detect a position in the direction of the longitudinal axis on the proximal end side of the transmission member.

19. The insertion device according to claim 9, wherein
an endoscope is configured by disposing an operation portion at a proximal end portion of the flexible tube.
